(19) United States
(12) Patent Application Publication
(10) Pub. No.: US 2025/0067717 A1
(43) Pub. Date: Feb. 27, 2025

(54) METHOD TO DESIGN SALT CAVERN FOR CYCLICAL WITHDRAWAL OF GAS

(71) Applicant: ARAMCO SERVICES COMPANY, Houston, TX (US)

(72) Inventors: Yanhui Han, Houston, TX (US); Younane N. Abousleiman, Norman, OK (US)

(73) Assignee: ARAMCO SERVICES COMPANY, Houston, TX (US)

(21) Appl. No.: 18/453,569

(22) Filed: Aug. 22, 2023

(51) Int. Cl. G01N 33/24; B65G 5/00; E21B 43/28; G01N 1/08; G06N 20/00

(52) U.S. Cl. CPC G01N 33/24; B65G 5/00; E21B 43/28; G01N 1/08; G06N 20/00; E21B 25/00

(57) ABSTRACT

Methods and systems include obtaining a correlation and obtaining a salt sample from a salt formation. The methods and systems further include, for each of N cycles, exposing, using an injection system, the salt sample to an nth amount of a gas and determining an nth value of a property by subjecting the salt sample, using an atomic force microscopy (AFM) system, to an AFM test. The methods and systems further include determining a relationship using the N values of the property, determining, using the correlation, a macroscale relationship based on the relationship, and generating a fit constitutive model by fitting the constitutive model to the macroscale relationship. The methods and systems include generating a model of a salt cavern within the salt formation based on the fit constitutive model and designing a salt cavern for withdrawal cycles of the gas using the model.

### BACKGROUND

As the world shifts from fossil fuels to renewable energy sources, the need to store renewable energy sources arises. Salt formations may be especially suited for storing renewable energy sources, such as hydrogen, as salt formations may be impermeable, water soluble, and self-healing. Salt formations may also possess useful thermo-mechanical properties. To store renewable energy sources within a salt formation, a salt cavern first needs to be mined within the salt formation.

Following mining of the salt cavern, renewable energy sources may be injected into, stored in, and/or withdrawn from the salt cavern. Injection, storage, and/or withdrawal may occur based on when the renewable energy sources are needed.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a method. The method includes obtaining a correlation and obtaining a salt sample from a salt formation. The method further includes, for each of N cycles, exposing, using an injection system, the salt sample to an nth amount of a gas and determining an nth value of a property by subjecting the salt sample, using an atomic force microscopy (AFM) system, to an AFM test. The method still further includes determining a relationship using the N values of the property, determining, using the correlation, a macroscale relationship based on the relationship, and generating a fit constitutive model by fitting the constitutive model to the macroscale relationship. The methods yet still further includes generating a model of a salt cavern within the salt formation based on the fit constitutive model and designing a salt cavern for withdrawal cycles of the gas using the model.

In general, in one aspect, embodiments relate to a system. The system includes an injection system, AFM system, and computer system. For each of N cycles, the injection system is configured to expose a salt sample from a salt formation to an nth amount of a gas and the AFM system is configured to determine an nth value of a property by subjecting the salt sample to an AFM test. The computer system is configured to determine a relationship using, at least in part, the N values of the property and determine, using a correlation, a macroscale relationship based on the relationship. The computer system is further configured to generate a fit constitutive model by fitting the constitutive model to the macroscale relationship, generate a model of a salt cavern within the salt formation based, at least in part, on the fit constitutive model, and designing a salt cavern for withdrawal cycles of the gas using, at least in part, the model.

Other aspects and advantages of the claimed subject matter will be apparent from the following description and the appended claims.

### BRIEF DESCRIPTION OF DRAWINGS

FIG. 1 illustrates salt caverns in accordance with one or more embodiments.

FIG. 2 illustrates a rock coring system in accordance with one or more embodiments.

FIG. 3 illustrates an injection system in accordance with one or more embodiments.

FIG. 4 illustrates an atomic force microscopy (AFM) system in accordance with one or more embodiments.

FIGS. 5A-5B illustrate a scanning electron microscope (SEM) image of a sample in accordance with one or more embodiments.

FIG. 6 describes a method in accordance with one or more embodiments.

FIG. 7 illustrates a mining system in accordance with one or more embodiments.

FIG. 8 illustrates a computer system in accordance with one or more embodiments.

FIG. 9 displays systems in accordance with one or more embodiments.

### DETAILED DESCRIPTION

In the following detailed description of embodiments of the disclosure, numerous specific details are set forth to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a sonic waveform” includes reference to one or more of such waveforms.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowchart may be omitted, repeated, and/or performed in a different order than the order shown. Accordingly, the scope disclosed herein should not be considered limited to the specific arrangement of steps shown in the flowchart.

Although multiple dependent claims are not introduced, it would be apparent to one of ordinary skill that the subject matter of the dependent claims of one or more embodiments may be combined with other dependent claims.

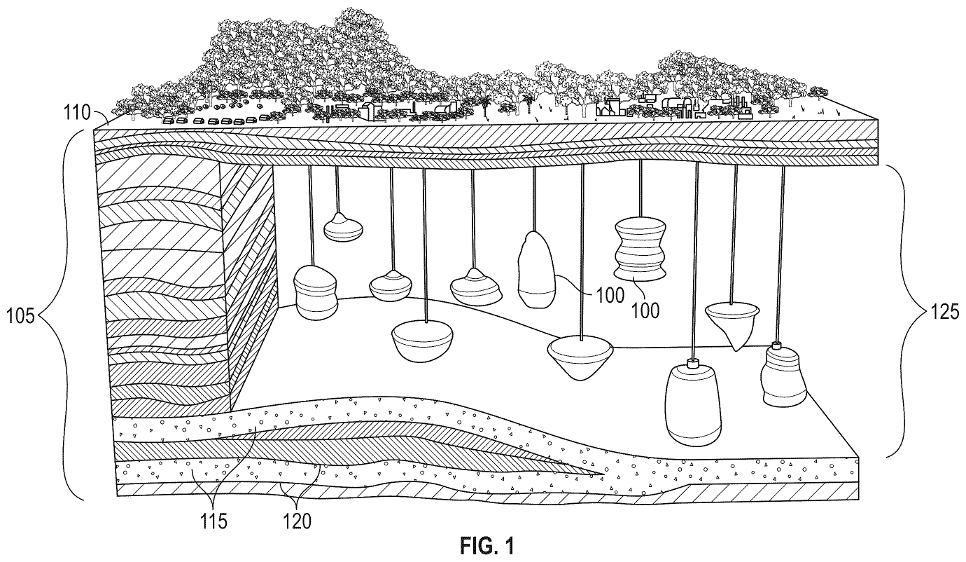

In the following description of FIGS. 1-9, any component described regarding a figure, in various embodiments disclosed herein, may be equivalent to one or more like-named components described regarding any other figure. For brevity, descriptions of these components will not be repeated regarding each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described regarding a corresponding like-named component in any other figure.

Methods and systems are disclosed to design a salt cavern to withstand withdrawal cycles of gas stored within the salt cavern. The salt cavern may be designed based on the use of systems such as an injection system, atomic force microscopy (AFM) system, and computer system. In some embodiments, the systems may further include a rock coring system, artificial intelligence (AI) model, and gas station. Further, the salt cavern may be designed based on a constitutive model and model.

FIG. 1 illustrates salt caverns 100 within a formation 105 in accordance with one or more embodiments. The formation 105 below the surface of the earth 110 may be made up of layers of rock 115 separated by geological boundaries 120. One or more layers of rock 115 within the formation 105 may be a salt dome, salt bed, or any other salt body. Hereinafter, a salt dome, salt bed, or any other salt body is generically referred to as a salt formation 125.

The salt formation 125 may be well suited for gas storage as the salt formation 125 may be impermeable, water soluble, and self-healing. To store gas in the salt formation 125, a manmade cavity known as a salt cavern 100 is mined within the salt formation 125 and gas injected into the salt cavern 100. In some embodiments, the salt cavern 100 may be considered a mini-salt cavern. In the context of this disclosure, gas may include, but is not limited to, natural gas, such as propane and methane, hydrogen, compressed air, derivatives thereof, and combinations thereof. The gas may be withdrawn from the salt cavern 100 as needed. However, to maintain sufficient pressure within the salt cavern 100 and/or allow expected withdrawal rates of the gas, a cushion gas (i.e., base gas) may also be injected into and permanently stored within the salt cavern 100 during the lifecycle of the salt cavern 100. The cushion gas may be, but is not limited to, nitrogen, methane, and carbon dioxide.

Storage of cushion gas and/or gas within the salt cavern 100 pressurizes the salt cavern 100 such that the walls of the salt cavern 100 experience local normal stresses and shear stresses (e.g., brushing shear stresses). Withdrawal of gas reduces the pressure of the salt cavern 100 and may cause the salt cavern 100 to shrink. Injection of gas increases the pressure of the salt cavern 100 and may cause the salt cavern 100 to expand. After tens to hundreds of thousands of injection and/or withdrawal cycles, the salt cavern 100 may be permanently expanded due to plastic deformation of the walls of the salt cavern 100. As such, cyclical injection and/or withdrawal of gas into/from the salt cavern 100 may threaten the stability and integrity of the salt cavern 100 and neighboring salt caverns 100. In turn, plastic deformation may lead to failure of one or more portions of the walls of the salt cavern 100 in the form of spalling where the salt cavern 100 partially caves in on itself.

To design a salt cavern 100 to withstand cyclical injection and/or withdrawal of gas, it may be advantageous to model a salt cavern 100 over the expected lifecycle, or a portion of the expected lifecycle, of the salt cavern 100 as the salt cavern 100 experiences injection and/or withdrawal cycles of gas. The salt cavern 100 may be a future salt cavern, partially-mined salt cavern, or currently-existing salt cavern. To model the salt cavern 100, a salt sample may need to be extracted from the salt formation 125. In some embodiments, a rock coring system may be configured to extract a salt sample from the salt formation 125.

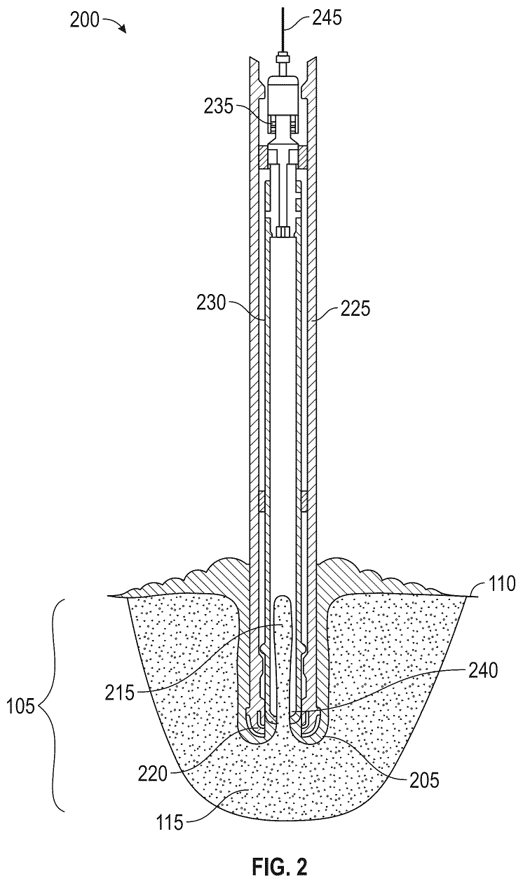

FIG. 2 illustrates a rock coring system 200 in accordance with one or more embodiments. The rock coring system 200 may be configured to simultaneously drill a well 205 within the formation 105 and retrieve one or more rock cores 215, including salt cores, along intervals of the well 205. As such, the rock coring system 200 may be considered part of a drilling system. The rock coring system 200 may collect rock cores 215 continuously or at intervals while drilling the well 205. To do so, the rock coring system 200 may include a coring bit 220 attached to a core barrel 225. Within the core barrel 225, an inner barrel 230 may be disposed between a swivel 235 attached to an upper portion of the core barrel 225 and a core catcher 240 disposed close to the coring bit 220. The coring bit 220 consists of an annular cutting or grinding surface configured to flake, gouge, grind, or wear away the formation 105 at the base or “toe” of the well 205. A central axial orifice may be configured to allow a cylindrical rock core 215 to pass through. The annular cutting surface of the coring bit 220 typically includes embedded polycrystalline compact diamond (PDC) cutting elements.

The inner barrel 230 within the core barrel 225 may be disposed above or behind the coring bit 220. Further, the inner barrel 230 may be separated from the coring bit 220 by the core catcher 240. As the coring bit 220 grinds away the formation 105, the cylindrical rock core 215 passes through the central orifice of the coring bit 220 and through the core catcher 240 into the inner barrel 230 as the coring bit 220 advances deeper into the formation 105. The inner barrel 230 may be attached by the swivel 235 to the remainder of the core barrel 225 to permit the inner barrel 230 to remain stationary as the core barrel 225 rotates together with the coring bit 220. When the inner barrel 230 is filled with the rock core 215, the core barrel 225 containing the rock core 215 may be raised and retrieved at the surface of the earth 110. The core catcher 240 serves to grip the bottom of the rock core 215 and, as lifting tension is applied to the drillstring 245 and the core barrel 225, the rock core 215 breaks away from the undrilled formation 105 below it. The core catcher 240 may retain the rock core 215 so that it does not fall out the bottom of the core barrel 225 through the annular orifice of the coring bit 220 as the core barrel 225 is raised to the surface of the earth 110.

In addition to collecting rock cores 215 while drilling the well 205, smaller “sidewall rock cores” may be obtained after drilling a portion or all of the well 205. A sidewall rock coring system (not shown) may be lowered by wireline into the well 205. When deployed, the sidewall rock coring system presses or clamps itself against the wall of the well 205 and a sidewall rock core is obtained either by drilling into the wall of the well 205 with a hollow coring bit 220 or by firing a hollow bullet into the wall of the well 205 using an explosive charge. More than 50 such sidewall rock cores may be obtained during a single deployment of a sidewall rock coring system into the well 205. Hereinafter, the term “rock coring system” is used to describe the rock coring system 200 as illustrated in FIG. 2 or the sidewall rock coring system.

Rock cores 215 collected along the portion of the well 205 that intersects the salt formation 125 are hereinafter referred to as salt cores. Salt cores provide representative samples of the salt formation 125. Further, salt cores permit physical examination and direct measurement and/or identification of salt properties in a laboratory setting. The analysis of salt cores in a laboratory setting may further provide evidence of how a mined salt cavern 100 will respond to injection and/or withdrawal cycles of gas stored in the salt cavern 100.

Under ideal circumstances, a salt core is recovered as a single, continuous, intact cylinder of the salt formation 125. However, frequently, the salt cores, as well as rock cores 215, take the form of several shorter cylindrical segments separated by breaks. The breaks may be a consequence of stresses experienced by the salt cores during coring or may be caused by pre-existing vugs, channels, and/or fractures within the salt formation 125.

In general, each extracted salt core may be up to 15 centimeters in diameter and approximately ten meters long. To prepare a salt core for testing in a laboratory setting, each salt core may be cut and ground into salt samples (e.g., salt plugs). Each salt sample may be in the shape of a cylinder (e.g., disc) or cuboid where each dimension is on the order of centimeters, though other shapes and dimensions may be used. Further, each salt core may be cut and ground along a particular axis of the well 205, such as parallel or perpendicular to the well 205.

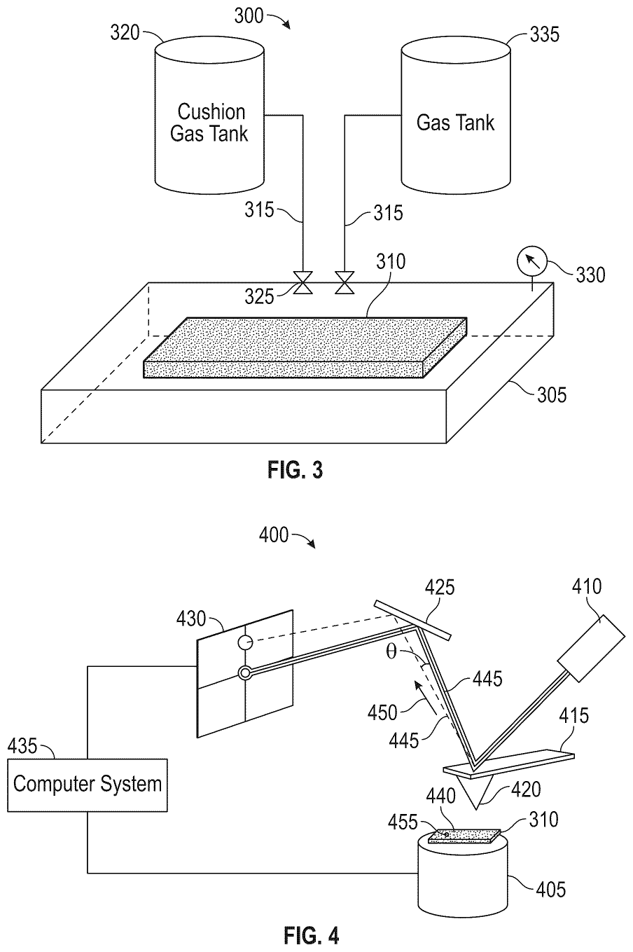

In the laboratory setting, the salt sample may be placed within an injection system. FIG. 3 illustrates an injection system 300 in accordance with one or more embodiments. The injection system 300 may include an injection chamber 305. The injection chamber 305 may be pressure and/or temperature controlled. In some embodiments, the pressure and/or temperature may mimic those of a salt cavern 100. Prior to injection, the salt sample 310 may be disposed within the injection chamber 305. In some embodiments, the injection system 300 may expose the salt sample 310 to cushion gas via a first flowline 315 coupled to a cushion gas tank 320. The cushion gas may only be permitted to flow unidirectionally towards and contact the salt sample 310 due to a first one-way flow control valve 325. Any amount of cushion gas may be exposed to the salt sample 310. Further, the pressure of the cushion gas in the injection chamber 305 may be displayed by a pressure gauge 330. The injection system 300 may also expose the salt sample 310 to gas via a second flowline 315 coupled to a gas tank 335. The gas may only be permitted to flow unidirectionally towards and contact the salt sample 310 due to a second one-way flow control valve 325. Any amount of gas may be exposed to the salt sample 310. Further, the pressure of the gas in the injection chamber 305 may be displayed by the pressure gauge 330. A person of ordinary skill in the art will appreciate that the salt sample 310 may see any number of cushion gas and/or gas exposures in any order without departing from the scope of the disclosure. For example, in some embodiments, the salt sample 310 may be exposed to only gas. In other embodiments, the salt sample 310 may be exposed to cushion gas followed by gas or vice versa. In still other embodiments, the salt sample 310 may be exposed to cushion gas once followed by any number of exposures to gas (or vice versa).

Following exposure, the salt sample 310 may be placed within an atomic force microscopy (AFM) system such that an AFM test may be performed on the surface of the salt sample 310. FIG. 4 illustrates an AFM system 400 in accordance with one or more embodiments. The AFM system 400 may be used to determine topographical, mineral, mechanical, electrical, magnetic, chemical, and/or optical properties (hereinafter generically “properties”) of the salt sample 310 on a microscale or smaller. As such, the AFM system 400 may be considered a high-resolution system.

The AFM system 400 may include a piezoelectric scanner 405 that the salt sample 310 is fixed to. The AFM system 400 may further include a laser generator 410, probe or cantilever 415 with tip 420, mirror 425, photodetector 430, and computer system 435. For illustration, the cantilever 415 with tip 420 are enlarged relative to the salt sample 310 in FIG. 4. In practice, the tip 420 contacts, intermittently contacts, or nearly contacts the surface 440 of the salt sample 310 during the AFM test. The tip 420 is located at the free end of the cantilever 415. The cantilever 415 may allow give (i.e., be flexible) and may have a known stiffness.

The AFM system 400 may rely on various non-destructive techniques to determine one or more properties of the salt sample 310. Each technique may rely on a primary mode of operation of the AFM system 400, a mode adjusted from a primary mode, and/or altered AFM hardware relative to the hardware illustrated in FIG. 4. For any technique, the piezoelectric scanner 405 moves the salt sample 310 such that the cantilever 415 moves across the surface 440 of the salt sample 310 in a raster pattern (i.e., raster scanning) during the AFM test.

The primary modes of the AFM system 400 may include static (i.e., contact) mode and dynamic mode. In static mode, the cantilever 415 contacts the surface 440 of the salt sample 310 during raster scanning. Dynamic mode may be further divided into tapping (i.e., intermittent) mode and non-contact mode. In tapping mode, the cantilever 415 oscillates, using a piezoelectric actuator (not shown), at or near the fundamental resonance frequency of the cantilever 415 during raster scanning. The fundamental resonance frequency may range from tens to hundreds of kilohertz (kHz). The tip 420 of the cantilever 415 is positioned near the surface 440 of the salt sample 310 such that the tip 420 lightly contacts the surface 440 of the salt sample 310 at the lowest amplitude of the oscillation. In non-contact mode, the cantilever 415 oscillates, using the piezoelectric actuator, at or near the fundamental resonance frequency of the cantilever 415 during raster scanning. The tip 420 of the cantilever 415 is positioned near the surface 440 of the salt sample 310 but does not contact the surface 440 of the salt sample 310 during oscillation.

For some techniques, the AFM test may be performed by emitting, from the laser generator 410, a laser beam 445 towards the cantilever 415 while the cantilever 415 operates in some mode. As the height of the surface 440 of the salt sample 310 changes during raster scanning, the position of the cantilever 415 may deflect based on the change in height of the surface 440 of the salt sample 310 and the stiffness of the cantilever 415. In turn, the deflection of the cantilever 415 changes the direction of propagation 450 of the laser beam 445 by some deflection angle θ. The deflected laser beam 445 may reflect off the mirror 425 and be detected by the photodetector 430. The photodetector 430 may convert the position of the detected laser beam 445 to an electric signal. The electric signal may be transmitted to and stored on a computer system 435 for further processing.

In the context of this disclosure, any technique may be used by the AFM system 400 to determine, directly or indirectly, one or more properties of the salt sample 310. Properties may include, but are not limited to, the height of the surface 440, mineral content, structure, stiffness, modulus (e.g., Young's modulus, shear modulus, and bulk modulus), resistivity, and porosity of the salt sample 310. A person of ordinary skill in the art will appreciate that this list is not meant to be exhaustive as tens of techniques may be performed using the AFM system 400.

For example, multiple techniques may be used to indirectly determine the stiffness of the salt sample 310. In some embodiments, force-displacement measurements may be determined from the AFM test based, at least in part, on the amount of deflection and the known stiffness of the cantilever 415 while in static mode. The stiffness may then be determined from the force-displacement measurements. In other embodiments, a technique known as pulsed force mode may be performed during the AFM test by oscillating the cantilever 415 at a frequency below the fundamental resonance frequency of the cantilever 415 to determine force-displacement measurements. The stiffness may then be determined from the force-displacement measurements. In still other embodiments, a technique known as contact resonance microscopy may be performed during the AFM test by using the cantilever 415 in contact mode and measuring the resonance frequency of the cantilever 415 to determine stiffness.

In some embodiments, one or more exposures of gas followed by any AFM test may be referred to as one cycle. In other embodiments, one or more exposures of cushion gas and one or more exposures of gas in turn (or vice versa) followed by any AFM test may be referred to as one cycle. N cycles may be performed on the salt sample 310, where N is an integer greater than or equal to two. However, in practice, tens to hundreds of cycles may be performed on the salt sample 310. Each of the N cycles may be indexed or counted from one to N using n. For example, if N=3, the nth cycle may refer to the first cycle, second cycle, or third cycle out of a total of three cycles. In some embodiments, the amount of cushion gas and/or gas exposed to the salt sample 310 during each cycle may be the same. In other embodiments, the amount of cushion gas and/or gas exposed to the salt sample 310 during each cycle may be different. As such, the amount of the cushion gas exposed to the salt sample 310 during the nth cycle is referred to as the nth amount of the cushion gas. Further, as such, the amount of the gas exposed to the salt sample 310 during the nth cycle is referred to as the nth amount of the gas. Further, the value of the property determined at one or more positions 455 on the surface 440 of the salt sample 310 during the AFM test during the nth cycle may be different. As such, the value of the property determined at one or more positions 455 on the surface 440 of the salt sample 310 during the nth cycle is referred to as the nth value of the property. A person of ordinary skill in the art will also appreciate that each nth value of the property may be a maximum, minimum, average, etc. of the nth value of the property at multiple positions 455 on the surface 440 of the salt sample 310.

The nth value of the property at multiple positions 455 on the surface 440 of the salt sample 310 for the nth cycle may be used to determine an nth image of the surface 440 of the salt sample 310. In some embodiments, the nth image may be an nth height image where each pixel within the nth height image is associated with a position 455 on the surface 440 of the salt sample 310 and the nth value of the height of the surface 440 (i.e., a property) of the salt sample 310 at the position 455. In other embodiments, the nth image may be an nth stiffness image where each pixel within the nth stiffness image is associated with a position 455 on the surface 440 of the salt sample 310 and the nth value of the stiffness (i.e., a property) associated with the position 455.

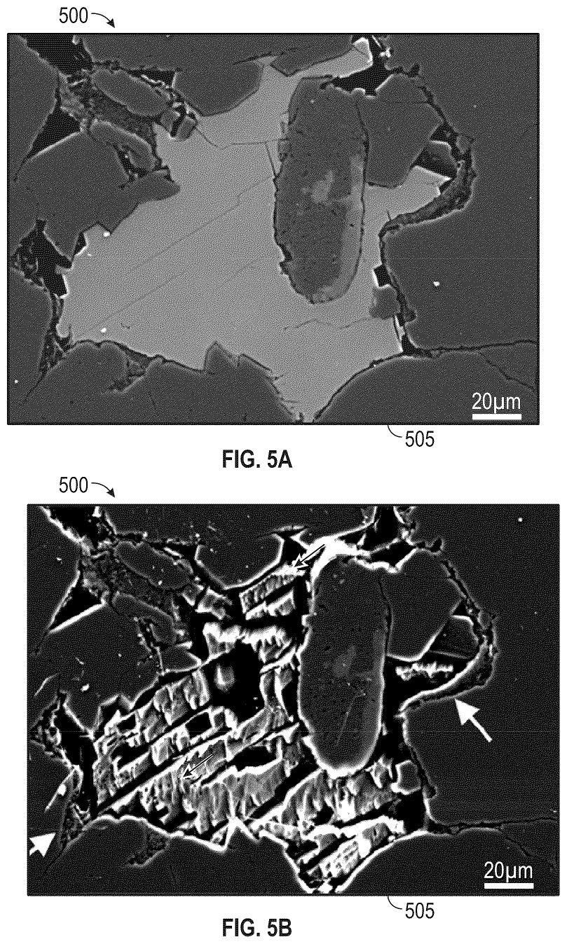

FIGS. 5A and 5B show a scanning electron microscope (SEM) image 500 of the surface 440 of a sample 505 before and after exposure to gas. Upon a comparison of FIGS. 5A and 5B, it is clear that the gas exposure has affected the structure, and thus one or more properties, of the sample 505.

The N values of the property at one or more positions 455 on the surface 440 of the salt sample 310 determined during the N cycles may be used to determine a relationship. In some embodiments, the relationship may relate the N values of the property to the N cycles. For example, the relationship may be a height relationship that relates the N values of the height of the surface 440 of the salt sample 310 at one or more positions to the N cycles. In another example, the relationship may be a stiffness relationship that relates the N values of the stiffness of the salt sample 310 at one or more positions to the N cycles. In other embodiments, the relationship may relate the N values of one property to N values of another property. For example, the relationship may be a force-displacement relationship that relates the N values of a force of the salt sample 310 to the N values of a displacement of the salt sample 310.

The one or more relationships determined following the N cycles may be associated to a microscale or smaller (e.g., nanoscale, atomic scale). As such, a correlation may be used to determine one or more macroscale relationships from the one or more relationships. In other words, the correlation is upscaling the relationship. In some embodiments, the correlation may be a regression that correlates the macroscale relationship to the relationship. The regression may take the form of, but is not limited to, a linear regression, exponential regression, quadratic regression, power regression, logarithmic regression, or combination thereof (i.e., a piecewise regression).

In other embodiments, the correlation may be determined using an artificial intelligence model. While a full discussion of artificial intelligence (AI) exceeds the scope of this disclosure, a brief overview of what AI is and what AI models may be used in the context of this disclosure is provided hereafter. AI, broadly defined, is the extraction of patterns and insights from data. The phrases “artificial intelligence.” “machine learning.” “deep learning.” and “pattern recognition” are often convoluted, interchanged, and used synonymously throughout the literature. This ambiguity arises because the field of “extracting patterns and insights from data” was developed simultaneously and disjointedly among a number of classical arts like mathematics, statistics, and computer science. For consistency, the term artificial intelligence is adopted herein, however, one skilled in the art will recognize that the concepts and methods noted hereafter are not limited by this choice of nomenclature.

AI model types may include, but are not limited to, k-means, k-nearest neighbors, neural networks, logistic regression, decision tree, random forests, extra trees, gradient boosted trees, support vector machine, generalized linear models, and Bayesian regression. Also, AI encompasses model types that may further be categorized as “supervised.” “unsupervised,” “semi-supervised,” or “reinforcement” AI models. One with ordinary skill in the art will appreciate that additional or alternate AI model categorizations may be defined without departing from the scope of this disclosure. AI model types are usually associated with additional “hyperparameters” which further describe the AI model. For example, hyperparameters providing further detail about a neural network may include, but are not limited to, the number of layers in the neural network, choice of activation functions, inclusion of batch normalization layers, and regularization strength. Commonly, in the literature, the selection of hyperparameters surrounding an AI model is referred to as selecting the AI model “architecture.”

Supervised AI models may require training using training data. In the context of this disclosure, training data may include AFM data and macroscale data. The AFM data may include any value of any property determined during any AFM test for any salt sample 310. The macroscale data may be data collected from macroscale tests performed on any salt sample 310 in a laboratory setting. Macroscale tests may include macroscale mechanical tests and macroscale imaging techniques, which may or may not be optical imaging techniques. Macroscale mechanical tests may include, but are not limited to, tension tests, compression tests, shear tests, dynamic cyclical tests, and failure tests. A person of ordinary skill in the art will appreciate that certain macroscale imaging techniques may be performed during a macroscale mechanical test without departing from the scope of the disclosure. Once the training data is acquired, the AI model may be trained to produce the macroscale relationship from the relationship by inputting the relationship into the trained AI model.

One or more macroscale relationships may be used to generate a fit constitutive model by fitting the constitutive model to the macroscale relationship. In the context of this disclosure, a constitutive model may be defined as a mathematical framework that may relate macroscale structural behavior to macroscale mechanical behavior. In turn, the macroscale mechanical behavior may be predicted from the macroscale structural behavior. For example, in some embodiments, one or more strains, strain rates, pressures, temperatures, and/or chemical potentials may be used to predict the stress of the walls of the salt cavern 100 using a fit constitutive model. The constitutive model may be considered phenomenological, structural, or in between. The constitutive model may include, but is not limited to, a Hou/Lux model, Lubby2 model, or any extensions/modifications thereof. However, the constitutive model may be any macroscale constitutive model that models the elasticity, plasticity, viscoelasticity, and/or failure of salt within the salt formation 125. Further, the constitutive model may include or be modified to account for time dependent mechanical properties such as creep and/or stress relaxation.

The constitutive model may be fit to the macroscale relationship using any method known to a person of ordinary skill in the art without departing from the scope of the disclosure. For example, in some embodiments, a least squares fitting method may be used to fit the constitutive model to the macroscale relationship by determining one or more constants within the constitutive model.

The fit constitutive model may be implemented within a model located on a memory of a computer system 435 in the form of software. The model may be, but is not limited to, a finite element (FE) model, finite volume model, or finite difference model. The modeling software may allow a user to model the salt formation 125 with salt cavern 100 in virtual three-dimensional space and time. The modeling software may then be used to generate a mesh over the modeled salt formation 125 to discretize the modeled salt formation 125 into thousands to hundreds of thousands of elements or volumes, for example. How each element or volume changes as modeled gas is injected into and/or withdrawn from the modeled salt cavern 100 may be controlled by the fit constitutive model and boundary conditions as well as other known mechanical properties of the salt. In some embodiments, boundary conditions may be known or estimated. The fit constitutive model for each element or volume may be organized into a system of equations that may be approximated at discrete points in time using numerical methods. The points in time may be on the order of seconds, days, months, or even years. In some embodiments, the modeling software may display a movie of how the salt formation 125 and salt cavern 100 change structurally and mechanically during injection and/or withdrawal cycles of gas over time.

A shear strength reduction (SSR) method may be used to determine the stability of the modeled salt formation 125 with salt cavern 100, in the form of a factor-of-safety (FOS), as the modeled salt cavern 100 experiences injection and/or withdrawal cycles of the gas. The SSR method may be an iterative method used to determine if the modeled salt formation 125 and salt cavern 100 have reached equilibrium based on reduced shear strength parameters. The ratio of the initial shear strength parameters and the reduced shear strength parameters may be the FOS.

The model may be used to design the salt cavern 100 to withstand injection and/or withdrawal cycles that the actual mined salt cavern 100 may see while avoiding non-sustainable large deformations (e.g., plastic and/or catastrophic deformations) of the salt cavern 100 that may compromise the integrity of the salt cavern 100. In some embodiments, the salt cavern 100 may be designed to withstand high frequency withdrawal cycles of gas. For example, high frequency withdrawal cycles of gas may occur when the salt cavern 100 stores hydrogen gas and is coupled to a hydrogen gas station. In these applications, fuel cell electric vehicles (FCEVs) may withdrawal the hydrogen gas from the mined salt cavern 100 to be used as fuel every few minutes. However, a person of ordinary skill in the art will appreciate that the mined salt cavern 100 may be designed to store gas other than hydrogen, for mid-to-low frequency withdrawal cycles of gas, and for applications apart from FCEV without departing from the scope of the disclosure.

The design of the salt cavern 100 may include geometric (i.e., structural), mechanical, electrical, magnetic, chemical, and/or any other specifications and considerations that may affect the ability of the salt cavern 100 to safely withstand injection and/or withdrawal cycles of the gas. In some embodiments, the design of the salt cavern 100 may further include considerations that may affect the ability of the salt cavern 100 to withstand high frequency injection cycles of hydrogen gas. Geometric specifications may include, but are not limited to, determining the initial position, which includes the initial depth, and initial dimensions of the salt cavern 100 within the salt formation 125, the volume of cushion gas to be initially and permanently stored within the salt cavern 100, and the volumetric window of the gas to be stored in the salt cavern 100. In some embodiments, the initial depth of the salt cavern 100 may be on the order of hundreds to thousands of meters below the surface of the earth 110. In some embodiments, the initial dimensions of the salt cavern 100 may form a volume on the order of hundreds of thousands to millions of cubic meters. Mechanical specifications may include, by are not limited to, determining the pressure window to maintain within the salt cavern 100, the maximum withdrawal cycle rate of the gas the salt cavern 100 can withstand, and the maximum number of injection and/or withdrawal cycles the salt cavern 100 can withstand. In some embodiments, the pressure window may be on the order of hundreds to thousands of pounds per square inch (psi). The pressure window may accommodate pressure fluctuations within the salt cavern 100 ranging from hundreds of psi seen during injection and/or withdrawal. Further, the pressure window may ensure the local normal and shear stresses placed on the walls of the salt cavern 100 cause primarily only elastic deformation of the salt. A person of ordinary skill in the art will appreciate that the design of the salt cavern 100 may include any specification that may ensure that the walls of the salt cavern 100 primarily function within the elastic range with little-to-no plastic deformation and/or failure, which could compromise the integrity of the salt cavern 100 and lead to spalling.

In some embodiments, the design of the salt cavern 100 may further include a mining plan, which may include a drilling plan, completion plan, and/or a portion thereof. The mining plan may be used to mine the salt cavern 100 to meet one or more of the design specifications and/or considerations. The mining plan may include how the well 205 that penetrates the salt formation 125 is drilled, what method of mining and/or circulation to use to mine the salt cavern 100, how to complete the salt cavern 100, and how to inject and/or withdrawal any fluid (e.g., gas, cushion gas, blanket solution, dissolving fluid, solution) into/from the salt cavern 100.

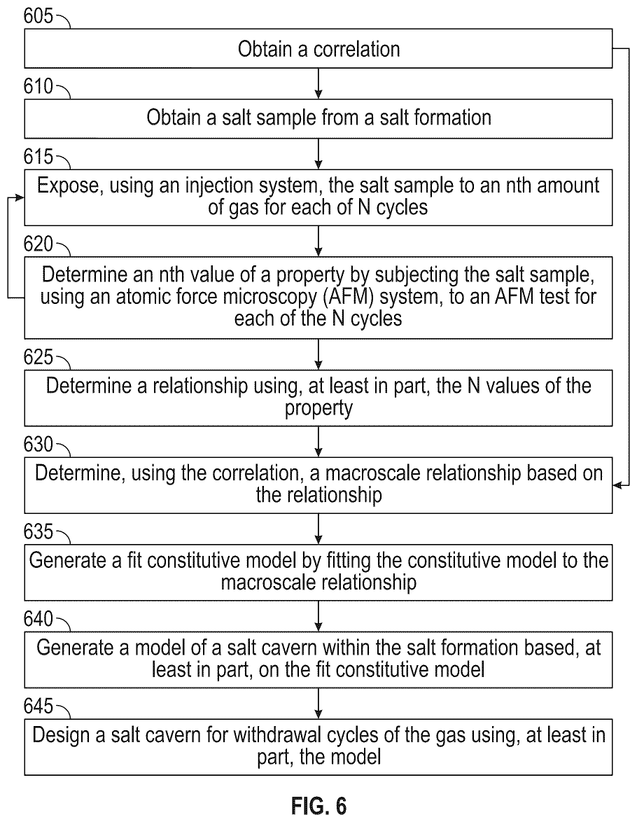

FIG. 6 describes a method of designing a salt cavern 100 in accordance with one or more embodiments. In step 605, a correlation is obtained. In some embodiments, the correlation may correlate a relationship, associated with a microscale or smaller, to a macroscale relationship. The correlation may be determined using regression analysis or an AI model as previously described.

In step 610, a salt sample 310 is obtained from a salt formation 125. The salt sample 310 may come from a salt core obtained from the salt formation 125 using a rock coring system 200 as previously described relative to FIG. 2.

Steps 615 and 620 are performed for each of N cycles, where N is an integer greater than or equal to two. Each of the N cycles may be indexed or counted from one to N using n.

In step 615, for each of the N cycles (i.e., the nth cycle), the salt sample 310 is exposed to an nth amount of a gas. Recall that the gas may include, but is not limited to, natural gas, such as propane and methane, hydrogen, compressed air, derivatives thereof, and combinations thereof. The gas may be exposed to the salt sample 310 using an injection system 300 as previously described relative to FIG. 3. In some embodiments, the salt sample 310 may be exposed to the gas more than once. In other embodiments, the salt sample 310 may be exposed to cushion gas and the gas in turn (or vice versa). A person of ordinary skill in the art will appreciate that any amount of cushion gas and/or gas may be injected onto the salt sample 310 any number of times to mimic how the walls of a salt cavern 100 may be affected by the cushion gas and/or gas.

In step 620, for each of the N cycles (i.e., the nth cycle), the salt sample 310 is subjected to an AFM test using an AFM system 400 as previously described relative to FIG. 4. The AFM test may determine an nth value of a property associated with one or more positions 455 on the surface 440 of the salt sample 310. In some embodiments, the property may be a height at a position 455 on the surface 440 of the salt sample 310 or a stiffness associated with a position 455 on the surface 440 of the salt sample 310 among many others.

In step 625, a relationship is determined using, at least in part, the N values of the property. The relationship may be associated with a microscale or smaller. Further, the relationship may quantify how the property of the salt sample 310 changes over the N cycles of gas exposure.

In step 630, a macroscale relationship is determined using the relationship and the correlation. The macroscale relationship may quantify how the macroscale property of the salt sample 310 changes over the N cycles of gas exposure.

In step 635, a fit constitutive model is generated by fitting the constitutive model to the macroscale relationship. The constitutive model may relate macroscale properties to one another such that one can be predicted from the other. For example, the constitutive model may relate stress and strain to one another such that the stress of the salt sample 310 may be predicted from the strain (as well as other properties) of the salt sample 310. The constitutive model may contain constants, which are determined during the fitting process. Any fitting process known to a person of ordinary skill in the art may be used to fit the constitutive model to the macroscale relationship.

In step 640, a model of a salt cavern 100 within the salt formation 125 may be generated. The model may be, but is not limited to, an FE model, finite volume model, or finite difference model. The model may model the macroscale mechanical behavior of the salt cavern 100 as the salt cavern 100 experiences injection and/or withdrawal cycles of the gas stored in the salt cavern 100 over time.

In step 645, a salt cavern 100 is designed for injection and/or withdrawal cycles of the gas using, at least in part, the model. The design of the salt cavern 100 may include geometric, mechanical, electrical, magnetic, chemical, and/or any other specifications or considerations that may affect the ability of the salt cavern 100 to safely withstand injection and/or withdrawal cycles of the gas as previously described.

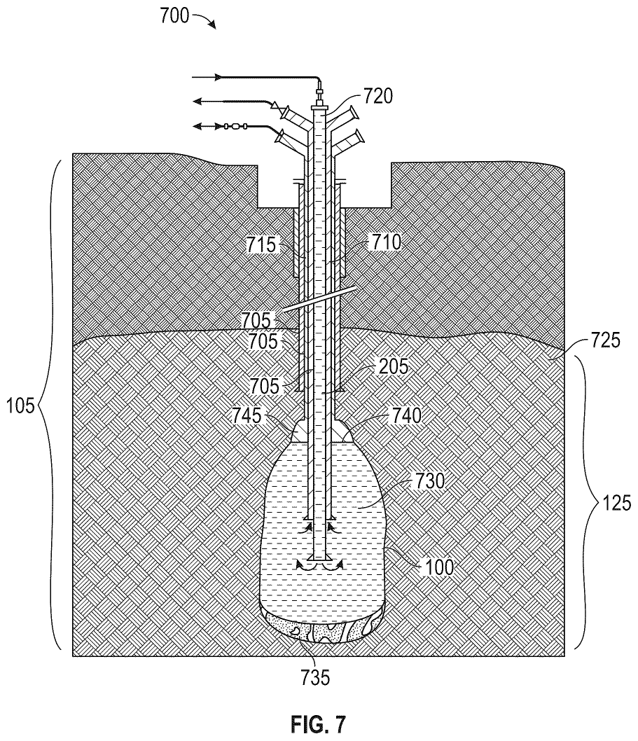

Following the design of the salt cavern 100 using the method described in FIG. 6, the salt cavern 100 may be mined within the salt formation 125 using a mining system. FIG. 7 illustrates a mining system 700 in accordance with one or more embodiments. The mining system 700 may be configured for mechanical mining or solution mining of the salt cavern 100 within the salt formation 125. FIG. 7 specifically illustrates a solution mining system configured for direct circulation, though reverse circulation could be used. Either circulation method may rely on nested casing 705 disposed along the previously-drilled well 205 to form an inner annulus 710 and outer annulus 715. During direct circulation, dissolving fluid 720, such as hot water, may be injected into the well 205 and salt formation 125. When the dissolving fluid 720 contacts the salt 725 within the salt formation 125, a solution 730 of dissolving fluid 720 and salt 725, such as brine, may be produced within the salt formation 125. The solution 730 of dissolving fluid 720 and salt 725 may be withdrawn from the salt formation 125 through the inner annulus 710. As this process of injection and withdrawal repeats, a salt cavern 100 may form. Insoluble materials 735 in the salt formation 125 may not dissolve in the dissolving fluid 720 but accumulate at the base of the salt cavern 100. Reverse circulation may be considered the opposite of direct circulation where the dissolving fluid 720 may be injected into the inner annulus 710 and the solution 730 of dissolving fluid 720 and salt 725 withdrawn from the well 205. Note that with reverse circulation, the dimensions of the salt cavern 100 may form differently than with direct circulation. Further, to prevent the salt cavern 100 from forming above a certain depth 740, a blanket solution 745 may be injected through the outer annulus 715 into the forming salt cavern 100. The water-insoluble blanket solution 745 may reside at the roof of the forming salt cavern 100 to protect the roof from dissolving during mining of the salt cavern 100. The process of injection and withdrawal may continue until the salt cavern 100 meets the designed dimensions.

Following mining and completion of the salt cavern 100, cushion gas and gas may be injected into the salt cavern 100 for storage. If the salt cavern 100 is to be used for high frequency withdrawals of hydrogen gas, a hydrogen gas station may be built on the surface of the earth 110 above the salt cavern 100. The hydrogen gas station may be coupled to the salt cavern 100 such that FCEVs may cyclically withdrawal hydrogen gas from the salt cavern 100 to be used as fuel every few minutes.

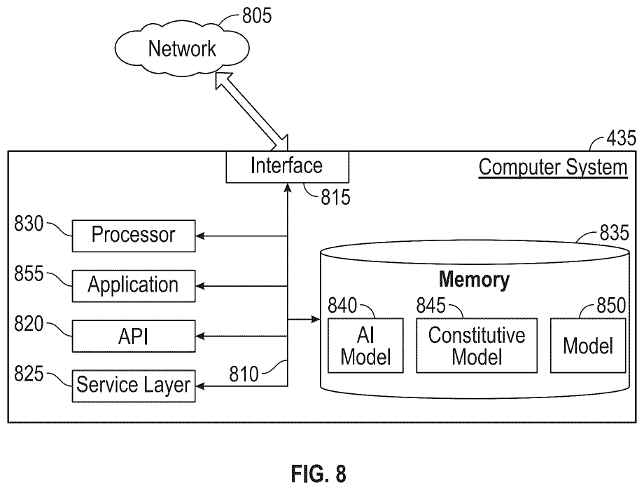

Steps 625, 630, 635, 640, and/or 645 of the method described in FIG. 6 may be performed by the computer system 435. FIG. 8 depicts a block diagram of a computer system 435 (hereinafter also “computer”) used to provide computational functionalities associated with described AI models, algorithms, methods, functions, processes, flows, procedures, constitutive models, and models as described in this disclosure, according to one or more embodiments. The illustrated computer 435 is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer 435 may include a computer that includes an input device, such as a keypad, keyboard, touch screen, or other device that can accept user information, and an output device that conveys information associated with the operation of the computer 435, including digital data, visual, or audio information (or a combination of information), or a GUI.

The computer 435 can serve in a role as a client, network component, a server, a database or other persistency, or any other component (or a combination of roles) of a computer system for performing the subject matter described in the instant disclosure. The illustrated computer 435 is communicably coupled with a network 805. In some implementations, one or more components of the computer 435 may be configured to operate within environments, including cloud-computing-based, local, global, or other environment (or a combination of environments).

At a high level, the computer 435 is an electronic computing device operable to receive, transmit, process, store, or manage data and information associated with the described subject matter. According to some implementations, the computer 435 may also include or be communicably coupled with an application server, e-mail server, web server, caching server, streaming data server, business intelligence (BI) server, or other server (or a combination of servers).

The computer 435 can receive requests over network 805 from a client application (for example, executing on another computer 435) and responding to the received requests by processing the said requests in an appropriate software application. In addition, requests may also be sent to the computer 435 from internal users (for example, from a command console or by other appropriate access method), external or third-parties, other automated applications, as well as any other appropriate entities, individuals, systems, or computers.

Each of the components of the computer 435 can communicate using a system bus 810. In some implementations, any or all of the components of the computer 435, both hardware or software (or a combination of hardware and software), may interface with each other or the interface 815 (or a combination of both) over the system bus 810 using an application programming interface (API) 820 or a service layer 825 (or a combination of the API 820 and service layer 825). The API 820 may include specifications for routines, data structures, and object classes. The API 820 may be either computer-language independent or dependent and refer to a complete interface, a single function, or even a set of APIs. The service layer 825 provides software services to the computer 435 or other components (whether or not illustrated) that are communicably coupled to the computer 435. The functionality of the computer 435 may be accessible for all service consumers using this service layer. Software services, such as those provided by the service layer 825, provide reusable, defined business functionalities through a defined interface. For example, the interface may be software written in JAVA, C++, or other suitable language providing data in extensible markup language (XML) format or another suitable format. While illustrated as an integrated component of the computer 435, alternative implementations may illustrate the API 820 or the service layer 825 as stand-alone components in relation to other components of the computer 435 or other components (whether or not illustrated) that are communicably coupled to the computer 435. Moreover, any or all parts of the API 820 or the service layer 825 may be implemented as child or sub-modules of another software module, enterprise application, or hardware module without departing from the scope of this disclosure.

The computer 435 includes an interface 815. Although illustrated as a single interface 815 in FIG. 8, two or more interfaces 815 may be used according to particular needs, desires, or particular implementations of the computer 435. The interface 815 is used by the computer 435 for communicating with other systems in a distributed environment that are connected to the network 805. Generally, the interface 815 includes logic encoded in software or hardware (or a combination of software and hardware) and operable to communicate with the network 805. More specifically, the interface 815 may include software supporting one or more communication protocols associated with communications such that the network 805 or interface's hardware is operable to communicate physical signals within and outside of the illustrated computer 435.

The computer 435 includes at least one computer processor 830. Although illustrated as a single computer processor 830 in FIG. 8, two or more processors may be used according to particular needs, desires, or particular implementations of the computer 435. Generally, the computer processor 830 executes instructions and manipulates data to perform the operations of the computer 435 and any algorithms, methods, functions, processes, flows, and procedures as described in the instant disclosure.

The computer 435 also includes a memory 835 that holds data for the computer 435 or other components (or a combination of both) that can be connected to the network 805. For example, in some embodiments, the memory 835 may store the AI model 840, constitutive model 845, and/or model 850. Although illustrated as a single memory 835 in FIG. 8, two or more memories may be used according to particular needs, desires, or particular implementations of the computer 435 and the described functionality. While memory 835 is illustrated as an integral component of the computer 435, in alternative implementations, memory 835 can be external to the computer 435.

The application 855 is an algorithmic software engine providing functionality according to particular needs, desires, or particular implementations of the computer 435, particularly with respect to functionality described in this disclosure. For example, application 855 can serve as one or more components, modules, applications, etc. Further, although illustrated as a single application 855, the application 855 may be implemented as multiple applications 855 on the computer 435. In addition, although illustrated as integral to the computer 435, in alternative implementations, the application 855 can be external to the computer 435.

There may be any number of computers 435 associated with, or external to, a computer system containing a computer 435, wherein each computer 435 communicates over network 805. Further, the term “client,” “user,” and other appropriate terminology may be used interchangeably as appropriate without departing from the scope of this disclosure. Moreover, this disclosure contemplates that many users may use one computer 435, or that one user may use multiple computers 435.

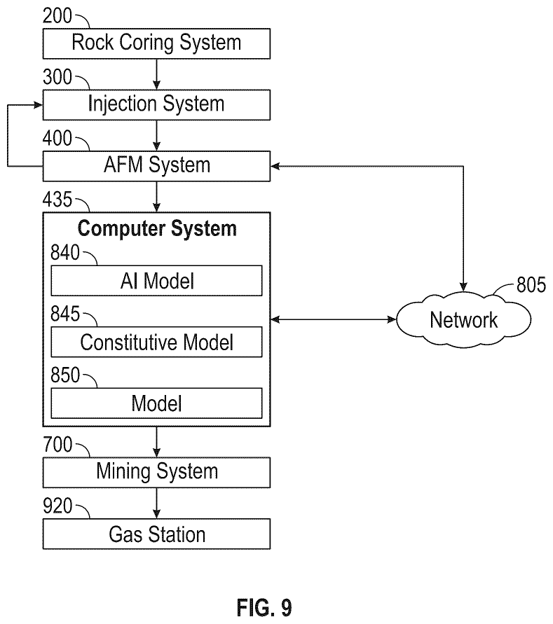

FIG. 9 shows systems in accordance with one or more embodiments. FIG. 9 recapitulates the systems that may be used to perform various processes described in the disclosure. The rock coring system 200 described in FIG. 2 may be configured to extract a salt core from a salt formation 125. The salt core may be cut and ground into one or more salt samples 310. Each salt sample 310 may be placed within the injection system 300 described in FIG. 3. The injection system 300 may be configured to expose the salt sample 310 to an nth amount of gas and, in some embodiments, an nth amount of cushion gas. Each salt sample 310 may be placed within the AFM system 400 described in FIG. 4. The AFM system 400 may be configured to perform an AFM test on the salt sample 310 to determine an nth value of a property. The injection system 300 and AFM system 400 may cyclically expose the salt sample 310 to an nth amount of the gas and AFM test the salt sample 310 for N cycles to determine N values of the property. The N values of the property may then be transferred to and stored on the memory 835 of the computer system 435 described in FIG. 8. In some embodiments, the AI model 840, constitutive model 845, and/or model 850 may be stored on the memory 835 of the computer system 435. In these embodiments, the computer system 435 may include a host computer communicably coupled via the network 805 to a supercomputer or cluster. In these embodiments, the supercomputer or cluster may train the AI model 840 and/or approximate the system of equations of the model 850. The computer system 435 may be configured to perform the steps 625, 630, 635, 640, and/or 645 of the method described in FIG. 6 to design the salt cavern 100 for injection and/or withdrawal cycles of the gas. The designed salt cavern 100 may be transferred to and stored on the mining system 700. The mining system 700 as described in FIG. 7 may be configured to mine the designed salt cavern 100 within the salt formation 125. Following mining and completion of the mined salt cavern 100, cushion gas and/or gas may be injected into the mined salt cavern 100 for storage. In some embodiments, the mined salt cavern 100 may be coupled to a gas station 920 such that the gas station 920 may frequently withdrawal the gas stored in the mined salt cavern 100 for use as fuel.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A method comprising:
obtaining a correlation;
obtaining a salt sample from a salt formation;
for each of N cycles:
exposing, using an injection system, the salt sample to an nth amount of a gas, and
determining an nth value of a property by subjecting the salt sample, using an atomic force microscopy (AFM) system, to an AFM test,
wherein N is an integer greater than or equal to two, and
wherein n is a count from one to N;
determining a relationship using, at least in part, the N values of the property;
determining, using the correlation, a macroscale relationship based on the relationship;
generating a fit constitutive model by fitting the constitutive model to the macroscale relationship;
generating a model of a salt cavern within the salt formation based, at least in part, on the fit constitutive model; and
designing a salt cavern for withdrawal cycles of the gas using, at least in part, the model.

2. The method of claim 1, further comprising mining, using a mining system, the salt cavern within the salt formation based, at least in part, on the designed salt cavern.

3. The method of claim 2, further comprising:
for each of the withdrawal cycles:
withdrawing, using a gas station, an amount of the gas from the mined salt cavern.

4. The method of claim 1, wherein exposing the salt sample further comprises exposing, using the injection system, the salt sample to an nth amount of a cushion gas.

5. The method of claim 1, wherein the gas comprises hydrogen gas.

6. The method of claim 1, wherein obtaining the correlation comprises:
obtaining training data comprising AFM data and macroscale data; and
training an artificial intelligence (AI) model using the training data,
wherein the AI model is trained to produce the macroscale relationship from the relationship.

7. The method of claim 1, wherein the property is associated to a microscale or smaller.

8. The method of claim 1, wherein the property comprises stiffness.

9. The method of claim 1, wherein determining the nth value of the property comprises an nth image, and
wherein the nth image comprises the nth value of the property at each of a plurality of positions on a surface of the salt sample.

10. The method of claim 1, wherein the model comprises a finite element (FE) model.

11. The method of claim 1, wherein designing the salt cavern comprises determining a position and dimensions of the salt cavern.

12. The method of claim 1, wherein designing the salt cavern comprises determining a maximum withdrawal cycle rate.

13. The method of claim 1, wherein designing the salt cavern comprises determining a pressure window.

14. The method of claim 1, wherein designing the salt cavern comprises applying a shear strength reduction (SSR) method to the model.

15. A system comprising:
for each of N cycles:
an injection system configured to:
expose a salt sample from a salt formation to an nth amount of a gas, and
an atomic force microscopy (AFM) system configured to:
determine an nth value of a property by subjecting the salt sample to an AFM test,
wherein N is an integer greater than or equal to two, and
wherein n is a count from one to N;
a computer system configured to:
determine a relationship using, at least in part, the N values of the property,
determine, using a correlation, a macroscale relationship based on the relationship,
generate a fit constitutive model by fitting the constitutive model to the macroscale relationship,
generate a model of a salt cavern within the salt formation based, at least in part, on the fit constitutive model, and
designing a salt cavern for withdrawal cycles of the gas using, at least in part, the model.

16. The system of claim 15, further comprising a mining system configured to mine the salt cavern within the salt formation based, at least in part, on the designed salt cavern.

17. The system of claim 16, further comprising a gas station configured to:
for each of the withdrawal cycles:
withdraw an amount of the gas from the mined salt cavern.

18. The system of claim 15, further comprising an artificial intelligence (AI) model configured to determine the correlation.

19. The system of claim 18, wherein the computer system is further configured to:
receive training data comprising AFM data and macroscale data; and
train the AI model using the training data,
wherein the AI model is trained to produce the macroscale relationship from the relationship.

20. The system of claim 15, further comprising a rock coring system configured to extract the salt sample from the salt formation.
